(19) United States
(12) Patent Application Publication
(10) Pub. No.: US 2026/0130842 A1
(43) Pub. Date: May 14, 2026

(54) Skincare Formulations

(71) Applicant: Bharati Malani Formulations LLC, Pune (IN)

(72) Inventor: Bharati Bhagwan Malani, Pune (IN)

(73) Assignee: Bharati Malani Formulations LLC, Pune (IN)

(21) Appl. No.: 18/947,292

(22) Filed: Nov. 14, 2024

(51) Int. Cl. A61K 8/92; A61K 8/19; A61K 8/34; A61K 8/73; A61Q 19/00; A61Q 19/08

(52) U.S. Cl. CPC A61K 8/922; A61K 8/19; A61K 8/345; A61K 8/73; A61K 8/927; A61Q 19/00; A61Q 19/007; A61Q 19/08

(57) ABSTRACT

Cosmetic compositions and their methods of preparation are disclosed herein. Such cosmetic compositions include a refined plant-based fat product and one or more wood-pressed oils, in addition to other components. In addition to the refined plant-based fat product and the one or more wood-pressed, oils, the cosmetic compositions disclosed herein can include any or all of glycerin, demineralized water, xanthan gum, borax, beeswax, and whole sea salt.

A portion of the disclosure of this patent document contains material which is subject to copyright or trade dress protection. This patent document may show and/or describe matter that is or may become trade dress of the owner. The copyright and trade dress owner has no objection to the facsimile reproduction by anyone of the patent disclosure, as it appears in the Patent and Trademark Office patent files or records, but otherwise reserves all copyright and trade dress rights whatsoever.

### CLAIM OF PRIORITY

This application does not claim priority to any patent or patent application.

### FIELD OF THE EMBODIMENTS

The present disclosure relates to cosmetic compositions for use by humans and other mammals, and their methods of preparation.

### BACKGROUND

Many cosmetics and other beauty products that are used in the marketplace today contain very harsh chemicals and despite the inclusion of these harsh chemical still are not overly effective at their intended purposes. Additionally, many of these cosmetics and other beauty products also have poor absorption and are difficult to spread at room temperature.

Due to the inclusion of these harsh chemicals, many of these products cause skin to dry out, resulting not only in skin that can be dry, dull, scaly, scarred, wrinkled, itchy, patchy, and skin that is clogged with sebum. Additionally, the inclusion of these synthetic ingredients, while able to improve the shelf-stability of these products, such inclusion also introduces toxins and other synthetic components to the human body. For consumers who seek cosmetics and other beauty products that are all natural and will help aid their body as opposed to harm it, such products are not suitable.

Thus, there exists a need for cosmetic compositions which are all-natural, effective, and are easy to utilize at room temperature.

### SUMMARY

The present disclosure provides for various cosmetic compositions and corresponding methods of preparation. In an embodiment, a composition in accordance with the present disclosure includes a refined plant-based fat product, a plurality of wood-pressed oils, and one or more additives. In various embodiments, the plurality of wood-pressed oils are selected from coconut oil, sesame oil, essential oil, neroli oil, almond oil, lavender oil, tea tree oil, geranium oil, castor oil, frankincense oil, clove bud oil, spearmint oil, jojoba oil, aniseed oil, rose absolute oil, rose oil, clary sage oil, or black pepper oil. Embodiments exist where the one or more additives are any of glycerin, demineralized water, xanthan gum, borax, beeswax, and/or whole sea salt.

In some embodiments, the plurality of wood-pressed oils are coconut oil, sesame oil, and neroli oil. In such embodiments, the refined plant-based fat product can be present in amounts of 35% to 37% by weight (% w/w), the coconut oil can be present in amounts of 27% to 32% w/w, the sesame oil can be present in amounts of 31% to 36% w/w, and the neroli oil can be present in amounts of 0.5 to 5.5% w/w. In other embodiments where the same wood-pressed oils are utilized, the refined plant-based fat product can be present in amounts of 30% to 40% w/w, the coconut oil can be present in amounts of 20% to 25% w/w, the sesame oil can be present in amounts of 28.5% to 38.5% w/w, and the neroli oil can present in amounts of up to 10% w/w.

Embodiments exist where the plurality of wood-pressed oils are sesame oil, lavender oil, and tea tree oil, and where the one or more additives are glycerin, demineralized water, xanthan gum, and borax. In these embodiments, the refined plant-based fat product can be present in amounts of 4% to 9% w/w, the sesame oil can be present in amounts of 17% to 22% w/w, the lavender oil can be present in amounts of up to 5% w/w, the tea tree oil can be present in amounts of up to 5% w/w, the glycerin can be present in amounts of 17% to 22% w/w, the xanthan gum can be present in amounts of 0.5 to 5.5% w/w, the borax can be present in amounts of 0.5 to 5.5% w/w, and the demineralized water can be present in amounts of 43% to 48% w/w. In other embodiments which utilize these same wood-pressed oils and additives, the refined plant-based fat product can be present in amounts of 1.5% to 11.5% w/w, the sesame oil can be present in amounts of 14.5% to 24.5% w/w, the lavender oil can be present in amounts of up to 10% w/w, the tea tree oil can be present in amounts of up to 10% w/w, the glycerin can be present in amounts of 14.5% to 24.5% w/w, the xanthan gum can be present in amounts up to 10% w/w, the borax can be present in amounts of up to 10% w/w, and the demineralized water can be present in amounts of 41.5% to 51.5% w/w.

In some embodiments, the plurality of wood-pressed oils used in the cosmetic compositions in accordance with the present disclosure are sesame oil, almond oil, neroli oil and carrot seed oil. In these embodiments the refined plant-based fat product can be present in amounts of 4.5% to 9.5% w/w, the sesame oil can be present in amounts of 58% to 63% w/w, the almond oil can be present in amounts of 25.5% to 30.5% w/w, the neroli oil can be present in amounts of 0.2% to 5.2% w/w, and the carrot seed oil can be present in amounts of up to 5% w/w. In other embodiments which utilize the same wood-pressed oils, the refined plant-based fat product can be present in amounts of 2% to 12% w/w, the sesame oil can be present in amounts of 55.5% to 65.5% w/w, the almond oil can be present in amounts of 23% to 33% w/w, the neroli oil can be present in amounts of up to 10% w/w, and the carrot seed oil can be present in amounts of up to 10% w/w.

Other embodiments exist where the wood-pressed oils used are sesame oil and geranium oil, and the additive is beeswax. In such embodiments, the plurality of wood-pressed oils are sesame oil and geranium oil, the one or more additives is beeswax, the refined plant-based fat product is present in amounts of 17.5% to 22.5% w/w, the sesame oil can be present in amounts of 63.5% to 68.5% w/w, the geranium oil can be present in amounts of up to 5% w/w, the beeswax can be present in amounts of 10.84% to 15.84% w/w. In other embodiments which incorporate the same wood-pressed oils and additive, the refined plant-based fat product is present in amounts of 15% to 25% w/w, the sesame oil is present in amounts of 61% to 71% w/w, the geranium oil can be present in amounts of up to 10% w/w, the beeswax can be present in amounts of 8.34% to 18.34% w/w.

Still, other embodiments of the cosmetic composition in accordance with the present disclosure exist where the plurality of wood-pressed oils are sesame oil, almond oil, and geranium oil, the one or more additives are beeswax, demineralized water, borax, and xanthan gum. In some of these embodiments, the refined plant-based fat product can be present in amounts of 17.5% to 22.5% w/w, the sesame oil can be present in amounts of 17.5% to 22.5% w/w, the almond oil can be present in amounts of 17.5% to 22.5% w/w, the geranium oil can be present in amounts of up to 5% w/w, the beeswax can be present in amounts of 17.5% to 22.5% w/w, the demineralized water can be present in amounts of 55.5% to 60.5% w/w, the borax can be present in amounts of up to 5% w/w, and the xanthan gum can be present in amounts of up to 5% w/w. In some other of these embodiments, the refined plant-based fat product can be present in amounts of 15% to 25% w/w, the sesame oil can be present in amounts of 15% to 25% w/w, the almond oil can be present in amounts of 15% to 25% w/w, the geranium oil can be present in amounts of up to 10% w/w, the beeswax can be present in amounts of 15% to 25% w/w, the demineralized water can be present in amounts of 53% to 63% w/w, the borax can be present in amounts of up to 10% w/w, and the xanthan gum can be present in amounts of up to 10% w/w.

Embodiments exist where the plurality of wood-pressed oils are castor oil and frankincense oil, and the one or more additives can be beeswax. In some of these embodiments, the refined plant-based fat product can be present in amounts of 44.82% to 49.82% w/w, the castor oil can be present in amounts of 44.73% to 49.73% w/w, the frankincense oil can be present in amounts of up to 5% w/w, and the beeswax can be present in amounts of up to 5% w/w. In other of these embodiments, the refined plant-based fat product can be present in amounts of 42.32% to 52.32% w/w, the castor oil can be present in amounts of 42.23% to 52.23% w/w, the frankincense oil can be present in amounts of up to 10% w/w, and the beeswax can be present in amounts of up to 10% w/w.

Embodiments of the method of preparation of cosmetic compositions in accordance with the present disclosure begins by first forming a first mixture by combining an unrefined plant-based fat with a purified liquid, wherein the purified liquid comprises at least some water content. Then, this first mixture can be indirectly heated to a temperature in the range of 37 to 40 degree Celsius. The first mixture can then be filtered through a mesh strainer and then allowed to solidify by reducing the temperature such that a first layer of purified liquid forms and a second layer forms containing the refined plant-based far product. In some embodiments, the mesh strainer has a mesh size in the range of 44 mm to 250 mm. The method can then proceed to the step of melting this mixture such that the first layer is separated from the second layer, utilizing the different melting points between the layers. From there, the remaining second layer can be cut into multiple pieces and then placed within a fabric package. This package can then be allowed to rest at a temperature of 1 to 4 degree Celsius for a period of at least 24 hours. Embodiments of the method then proceed to the step of liquefying the refined plant-based far product by warming the product to a temperature of 100 degrees Celsius. The method can then proceed to the step of combining at least one oil, which can be a wood-pressed oil or other type of plant-based oil, with the refined plant-based fat product by stirring. In various embodiments these methods can be repeated until the resulting product has a substantially homogeneous color. In some embodiments, the cosmetic compositions in accordance with the present disclosure have a water content less than or equal to 0.2%.

In various embodiments, the purified liquid in accordance with the present disclosure is pretreated using one or more of chemical treatment, iodine treatment, chlorine treatment, filtration, ultraviolet light, and/or boiling. Embodiments exist where the purified liquid in accordance with the present disclosure has a water concentration of greater than 50%. In some embodiments, the unrefined plant-based fat and the purified liquid are combined in a ratio of 4:1. In various embodiments, the plant-based far is either kokum butter, shea butter, cocoa butter, coconut oil, or mango butter.

It should be noted that cosmetic compositions in accordance with the present disclosure may be safely consumed by humans or other mammals.

### DETAILED DESCRIPTION

Reference will now be made in detail to each embodiment of the present invention. Such embodiments are provided by way of explanation of the present invention, which is not intended to be limited thereto in any manner whatsoever. In fact, those of ordinary skill in the art may appreciate upon reading the present specification and viewing the present drawings that various modifications and variations can be made thereto.

For purposes of the present disclosure of the invention, unless specifically disclaimed, the singular includes the plural and vice-versa, the words “and” and “or” shall be both conjunctive and disjunctive, the words “any” and “all” shall both mean “any and all”.

The present disclosure provides for various cosmetic compositions which are suitable for various purposes. While different embodiments in accordance with the present disclosure have different use cases, common elements between these embodiments do exist. Namely, such elements are the inclusion of a refined plant-based fat such as kokum butter, shea butter, cocoa butter, coconut oil, or mango butter. These plant-based fats are beneficial because they are all-natural and non-toxic ingredients. However, it should be noted that while the embodiments in accordance with the present disclosure do use refined plant-based fat, such refining is not done an in industrial manner, which uses chemicals. In these cases, the raw kokum butter is heated to high temperatures, sometimes boiled, and passed through a range of sieves of large holes to very fine. Additionally, solvents are used to refine kokum butter and bleach is added to make the end-product white. All these methods remove the benefits of the unrefined kokum butter as solvents and bleach just add toxic chemicals to the end product. The end-product of these processes is an ineffective white butter, with little smell.

Instead, an all-natural refinement method, further detailed herein, is used such that the refined plant-based fat product contains no synthetic chemicals, providing an all-natural foundation for the cosmetic compositions in accordance with the present disclosure to be formulated. These plant-based fats have a high concentrations of fatty acids, such as omega-3 and omega-6 as well as high concentrations of Vitamin E, all of which contribute to the its non-comedogenic properties of the cosmetic compositions in accordance with the present disclosure, making them suitable for most skin types without clogging pores. In particular, these plant-based fats are particularly useful for their ability to lock in moisture while allowing the skin to breathe. Additionally, these refined plant-based fats have incredible emollient, nutritive, and astringent properties. When blended with other oils, the plant-based fats form cosmetic compositions in accordance with the present disclosure that penetrate the skin deeply while delivering nutrients to the epidermis and dermis layers, thus promoting healthy, glowing skin.

In various embodiments, the cosmetic compositions in accordance with the present disclosure can be formulated into a number of different products, such as serums, cleansing jellies, butters, creams, scrubs, balms, masks, and pomades. All of these products have the benefit of providing quick, efficient results, without introducing chemicals or other unwanted agents to the body. In particular, the embodiments in accordance with the present disclosure utilize plant-based fats, in concert with natural essential wood-pressed oils and other botanical extracts.

These compositions are engineered to provide deep hydration, restore skin elasticity, promote cell regeneration, and address specific dermatological concerns such as acne, wrinkles, and environmental damage. Despite minor variations in their ingredient ratios, the underlying preparation of each formulation follows similar processes. It should be noted that the essential oils are preferably wood-pressed such that said oils are also all-natural. Chemical-based extractions of these essential oils are not suitable for use in the cosmetic compositions in accordance with the present disclosure.

In an embodiment, the cosmetic composition in accordance with the present disclosure is a butter, which contains sesame oil, coconut oil, and naturally-refined kokum butter. Of note is the fact that the essential oils used in this embodiment have varying molecular sizes, which aids in the penetration of the lowest layers of skin which also comprise the smallest of pores. For optimal use, this embodiment can be applied after a bath or other washing to help lock in the moisture of such bath or washing. The quick absorption of this embodiment is beneficial for two reasons The first is that the skin gets moisturized quickly, and the second is that due to the varied size of the essential oils, the skin is permeated effectively such that vital nutrients and the oils themselves to the lower layers of the skin. Collectively, these features allow the skin of the user to heal quickly and to have a fresh vitality and radiance.

In one embodiment of preparing this particular cosmetic composition in accordance with the present disclosure begins by first refining the plant-based fat product, such as kokum butter. By weight, 80% unrefined kokum butter pieces are placed in a double boiler along with 20% of filtered water. This mixture is heated until all the kokum butter has melted completely (approximately 37-40 degrees Celsius). Care must be taken to avoid overheating as overheating the mixture will deplete nutrients from the kokum butter. The mixed liquid is then strained into a bowl using a strainer to remove the larger impurities. The liquid is then cooled to room temperature subsequently placed a freezer overnight, resulting in two distinct layers to form within the vessel, one of the layers being ice. The frozen mixture is then allowed to melt such that the ice layer melts away leaving a slab of clarified kokum butter. Occasionally, if there is a thin, dark layer at the bottom of the slab is present and must be cut off, as this layer consists of extremely fine dust particles that may not sink down to the water layer. By following the above procedure, the integrity of the components of the plant-based fat product is preserved. The resulting refined product is placed within a fabric bag and refrigerated for at least 24 hours. Then, a portion of the refined product is separated and re-melted and placed into a vessel with unfractionated, wood pressed coconut oil. From there, the solids in the coconut oil will melt due to the warmth of the vessel and refined kokum butter. If these solids do not melt heat must be applied to the vessel. Upon all the solids melting, the wood-pressed sesame oil is added and the entire mixture is allowed to partially cool so that the neroli essential oil can be safely added to the mixture, as adding the neroli when the mixture is hot will make the fragrance evaporate into the air. The liquid is then stirred well and poured into one or more final contains, which are then kept in the freezer overnight. The next day, the containers are transferred to the refrigerator and are ready to be used by the end-user.

In these embodiments and others, the use of sesame oil provides anti-oxidants to the composition which help combat free radicals and prevent premature aging by reducing wrinkles, fine lines, and age spots. Additionally, sesame oil can encourage supple and youthful looking skin by maintaining the skin's elasticity. Including coconut oil is beneficial as it also contains antioxidants and helps to increase the body's production of collagen, which assists with both skin regeneration and skin elasticity. Coconut essential oil contains a number of beneficial fatty acids, and by extracting the essential oil via wood pressing, the fatty acids contained therein are preserved for inclusion in the final composition. Such fatty acids include capric acid and lauric acid, which together are capable of killing acne-causing bacteria and other harmful microorganisms which grow on skin. The inclusion of coconut oil in the relevant embodiments also assists with the moisturizing properties of the composition, allowing such composition to reduce dry and cracked skin, as well as assist with fixing uneven skin tone.

In one embodiment, the cosmetic composition in accordance with the present disclosure combines sesame oil, coconut oil, and neroli essential oil alongside kokum butter. The blend of these ingredients creates a powerful moisturizer that penetrates the lower layers of a user's skin. Sesame oil can act as a hydrating agent, as it is rich in antioxidants like Vitamin E and sesamol, which help prevent premature aging by combating free radicals. In these embodiments, coconut oil complements these properties by improving skin elasticity and assisting in collagen production. Such embodiments are capable of addressing fine lines and wrinkles on the user's face while simultaneously acting as an antibacterial and antifungal agent. Neroli essential oil adds to the blend with its regenerative properties, enhancing skin cell renewal and elasticity. In particular, the antioxidant, anti-microbial and antibacterial characteristics of neroli treat scar tissue, acne, pimples, blemishes, pigmentation, whiteheads and blackheads. In various embodiments, the butter disclosed above comprises 32-37% kokum butter, 27-32% unfractionated, wood-pressed coconut oil, 31-36% wood press sesame oil, and 0.5-5.5% neroli essential oil. In other embodiments, said butter comprises 30-40% kokum butter 25-25% unfractionated, wood-pressed coconut oil, 28.5-38.5% wood press sesame oil, and 0-10% neroli essential oil.

Embodiments exist where the cosmetic composition in accordance with the present disclosure is a hair pomade. In these embodiments, the composition comprises jojoba oil, beeswax, sesame oil, refined kokum butter, and black pepper oil. One method of preparing this embodiments is to mis the kokum butter, jojoba oil, sesame oil and beeswax in a double boiler, and heating until all the solid components have melted. The vessel is then removed from the heat source and is mixed continuously until the blend starts to cool and thicken. However, it is important that this mixture not be cooled down too quickly otherwise the ultimate consistency will be off. Ideally, a honey-like viscosity should be achieved. From there, the wood-pressed black petter essential oil is added to the mixture and mixed again. This new mixture is then refrigerated until it is used by the end-user. In some embodiments, the disclosed pomade comprises 26.84-31.84% sesame oil, 13.8-18.8% kokum butter, 30-35% jojoba oil, 17.06-22.06% beeswax, and 0-5% black pepper oil. In other embodiments, the disclosed pomade comprises 24.34-34.34% sesame oil, 11.3-21.3% kokum butter, 27.5-37.5% jojoba oil, 14.56-24.56% beeswax, and 0-10% black pepper oil.

In these embodiments, the inclusion of jojoba oil is beneficial as it is rich in vitamins and minerals which nourish hair, including vitamin C, various B vitamins, vitamin E, copper, and zinc. Collectively, these elements can protect hair against dryness, breakage, and split ends. By moisturizing the scalp and is a remedy for dandruff, as well as being able to prevent hair loss and promote hair thickness by strengthening hair. Moreover, by moisturizing hair follicles, dryness that leads to hair loss is prevented. Sesame oil helps soothe the scalp. It can treat scalp itching and irritation, promote hair growth and thickness, condition the hair, and improve the general health of your scalp. The inclusion of beeswax in these embodiments provides a natural hold and helps create texture, making it easier to style hair. It also has emollient properties allowing it to effectively lock in moisture. When applied to the hair, it can prevent dryness by keeping the strands hydrated. Additionally, beeswax forms a protective barrier on the hair shaft, shielding it from environmental factors like wind, sun, and pollution. This can help prevent damage and split ends. Finally, beeswax can add a subtle shine to hair, making it look healthier and more lustrous.

When used herein, sesame oil encourages hair growth and prevents hair loss through the component element of seamin. Sesamin also has hair-darkening properties that help fight premature greying. Sesame oil also contains essential fatty acids, vitamin E, phosphorus, and other nutrients that strengthen your hair from within and can prevent breakage of the hair. Moreover, sesame oil has a cooling effect on the scalp and provides relief from scalp irritation. It also possesses antifungal properties that can fight dandruff and itching. Sesame oil helps soothe the scalp. It can treat scalp itching and irritation, promote hair growth and thickness, condition the hair, and improve the general health of the scalp. Refined kokum butter is non-comedogenic, meaning it won't create a build-up in the hair follicles. Therefore, this ingredient can leave the scalp refreshed and revitalised, without any heavy feeling in the roots. Kokum butter nourishes a dry scalp and corresponding hair strands. Kokum butter also provides intense hydration which supports and enhances hair elasticity, helping to prevent breakage. The inclusion of black pepper essential oil fights hair fall problems and promotes the growth of new hair as it contains piperine which can help hair regrow once it is lost. Additionally, black pepper oil improves scalp circulation, stimulates and strengthens follicles, increases cellular metabolism, and provides protection from root to tip. This oil detoxifies the scalp, controls dandruff and nourishes hair with Vitamin C.

Other embodiments exist where the cosmetic composition is a face mask that comprises a combination of cocoa butter, sesame oil, castor oil, kokum butter, and coconut oil. These ingredients are beneficial as they have hydrating and anti-aging properties. The combination of these ingredients results in an unexpectedly deeply nourishing mask that restores skin vitality Cocoa butter, which has a rich fatty acid content, provides intense hydration and helps reduce the appearance of scars by increasing skin elasticity and reducing the appearance of scars. It is also highly nourishing for every skin type and contains polyphenols which increase blood flow to the skin and slow down the aging process. Castor oil is beneficial to include as it has humectant properties which draw moisture from the air and ensures that the skin remains hydrated and supple, including reducing puffiness under the eyes. Additionally, castor oil contains triglycerides which moisturize the skin and help in removing dirt from the skin. Kokum butter, again, plays a key role by promoting cell regeneration and regulating oil secretion, making it suitable for those with acne-prone skin. Kokum butter is rich in vitamin E which mitigates sun damage. As noted above, the antioxidant constituents in kokum butter combat free radicals. Moreover, kokum butter promotes cell regeneration which helps in fading scars, hyperpigmentation and uneven skin tones, and assists with the regulation of oil secretions, effectively addressing breakouts, pimples, blackheads and whiteheads. It also tones and firms the skin while being gentle enough for sensitive skin. The anti-inflammatory properties encourage lightening, brightening and soothing effects even for the most sensitive skin types. Its anti-bacterial properties cleanse pores of impurities, germs and dust. Sesame oil is high in vitamin E which provides a natural sunscreen and protection from ultraviolet rays.

In some embodiments, the face mask in accordance with the present disclosure comprises 34.4-39.4% cocoa butter, 40.62-45.62% sesame oil, 5.2-10.2% castor oil, 6.7-11.7% kokum butter, and 0.58-5.58% coconut oil. In other embodiments, the face mask in accordance with the present disclosure comprises 31.9-41.9% cocoa butter, 38.12-48.12% sesame oil, 2.7-12.7% castor oil, 4.2-14.2% kokum butter, and 0-10% coconut oil.

Other embodiments exist where the cosmetic composition in accordance with the present disclosure is a night cream comprising refined kokum butter, almond oil, sesame oil, and rose absolute oil. The benefits of the inclusion of the refined kokum butter and the sesame oil have been disclosed above. Almond oil is beneficial to include as it has anti-inflammatory properties that can help reduce puffiness around the eyes. As it is high in vitamin E, this wood-pressed almost essential oil helps to nourish and moisturize the delicate skin around the eyes which leads to a reduction in any dark circles on that skin. Moreover, vitamin E is a powerful antioxidant that protects the skin from damage caused by free radicals. Additionally, almond oil contains vitamin A which helps reduce fine lines and wrinkles as well as Vitamin K which helps to erase dark spots and blemishes on the skin. This almond oil also contains vitamin C which helps to stimulate collagen production thereby helping improve skin elasticity and reduce the appearance of fine lines and wrinkles. Almond oil can also help reduce the appearance of sun damage. These embodiments also include rose absolute oil, which is high aromatic and is unexpectedly beneficial at dealing with broken capillaries, dry skin, wrinkles, eczema, scars, and sensitive complexions when used in connection with the other elements of these embodiments.

In some embodiments, the night cream in accordance with the present disclosure contains 20.84-25.84% almond oil, 24.16-29.16% kokum butter, 46.83-51.83% sesame oil, and 0-5% rose absolute oil. In other embodiments, the night cream in accordance with the present disclosure contains 18.34-28.34% almond oil, 21.66-31.66% kokum butter, 44.33-54.33% sesame oil, and 0-10% rose absolute oil. These embodiments are best applied by the end user at night just prior to bedtime. These embodiments can be thicker than the end-user desires. While this thickness allows these creams to coat, heal and protect the skin, if an end-user desires a more spreadable consistency is required sesame oil can be added, however this will reduce the efficacy of the cream.

Embodiments of the cosmetic compositions in accordance with the present disclosure exist where said composition is a nourishing cream comprising Kokum butter, almond oil, sesame oil, beeswax, demineralized water, xanthan gum, borax and geranium essential oil. The combination of these ingredients unexpectedly provides the skin with a large amount of both hydration and nutrition, thereby making the skin of an end-user healthier by fighting acne, removing impurities, fading blemishes, improving skin elasticity and texture, fighting off lines and wrinkles, and imparting a glow. The benefits of including refined kokum butter, almond oil, and sesame oil have been discussed above. Including beeswax in this composition is beneficial as beeswax forms a protective barrier from external factors and locks in moisture to prevent water loss, thereby helping skin stay soft and hydrated. Additionally, beeswax has skin-softening properties and enhances skin elasticity in addition to being a good source of vitamin A. Xanthan gum is included in this composition as it gives the composition a desired consistency, making it easier to spread and absorb into the skin. Xanthan gum also provides hydration, keeping the skin nourished while forming a fine film on the skin that helps to retain moisture for long hours post application. This fine film also shields the skin against harsh environmental pressures, hence soothing and calming the skin. Borax is a highly unconventional ingredient to include in cosmetic compositions for use with humans and other mammals. As part of this composition, borax serves to eradicate skin bacteria and can remove dead skin cells and excess oil. Finally, geranium oil is beneficial to include in such a composition as it reduces wrinkles and the signs of aging by calming and soothing irritated skin. Additionally it can reduce scar tissue and marks caused by skin irritation. It controls oiliness and balances sebum production and gives the composition a pleasant scent.

In such embodiments, the cosmetic composition is an emulsion of oil and water, where the kokum butter, almond oil, sesame oil, and beeswax form the oil phase which gets heated. Separately, demineralized water is heated and then the xanthan gum and borax are added to the water, however as xanthan gum is not water soluble it will just disperse within the liquid. The addition of these powders to the water-phase will cause it to thicken and take on a gel like consistency. This gives the finished emulsion a spreadable, fluid texture. Next, using an emulsifying machine, the oil phase is mixed on medium speed and gradually the water phase is added to the oil phase until a smooth emulsion forms. Once the emulsion is formed, it is continued to be mixed until cooled. Then the geranium oil is mixed in and the mixture is sealed in an air-tight container and allowed to rest overnight. It is then ready to be transferred to a different vessel and used by the end user.

Embodiments exist where the nourishing cream in accordance with the present disclosure comprises 17.5-22.5% kokum butter, 17.5-22.5% almond oil, 17.5-22.5% sesame oil, 17.5-22.5% beeswax, 55.5-60.5% demineralized water, 0-5% borax, 0-5% geranium oil, and 0-5% xanthan gum. In other embodiments, the nourishing cream in accordance with the present disclosure comprises 15-25% kokum butter, 15-25% almond oil, 15-25% sesame oil, 15-25% beeswax, 53-63% demineralized water, 0-10% borax, 0-10% geranium oil, and 0-10% xanthan gum.

In some embodiments of the cosmetic compositions in accordance with the present disclosure, the cosmetic composition is a foot scrub having anti-microbial, astringent, anti-fungal, anti-inflammatory, and antioxidant properties. In these embodiments, the food scrub comprises kokum butter, sesame oil, coarse sea salt, clove bud oil, and spearmint oil. In addition to the previously discussed benefits of kokum butter and sesame oil, the coarse sea salt is a detoxifying exfoliant for dry, rough, flaky skin. It not only helps sloughing off dead cells but also revitalizes the top layers of skin with minerals and vitamins. The inclusion of spearmint oil is beneficial as spearmint oil is anti-inflammatory, antioxidant, astringent, antiseptic and helps relieve pain. It has a warm, spicy-herbaceous minty aroma which is imparted onto the final product. Finally, the clove bud oil has a sweet-spicy fragrance with a fruity-fresh top note. It is antiseptic and an anti-fungal remedy which is effective against fungal infections like athlete's foot and candida.

In some of the embodiments of the foot scrub, said foot scrub comprises 28.4-33.4% kokum butter, 28.4-33.4% sesame oil, 34.05-39.05% coarse sea salt, 0-5% clove bud oil, and 0-5% spearmint oil. In other embodiments, the foot scrub comprises 25.9-35.9% kokum butter, 25.9-35.9% sesame oil, 31.55-41.55% coarse sea salt, 0-10% clove bud oil, and 0-10% spearmint oil.

Embodiments of the cosmetic compositions in accordance with the present disclosure also include embodiments where said cosmetic composition is a foot cream comprising kokum butter, castor oil, beeswax, and frankincense oil. The benefits of kokum butter, castor oil, and beeswax have been discussed above. The inclusion of frankincense oil is beneficial as it has anti-inflammatory, antiseptic, vulnerary, anti-microbial properties. Thus, in connection with the other components, this oil helps to eradicate infections and microbes while assisting cracks and wounds to heal. Collectively, these components come together to provide the unexpected benefits of a complete solution for footcare in a single cream.

In some embodiments of this foot cream, the foot cream comprises 44.82-49.82 kokum butter, 44.73-49.73% castor oil, 0-5% beeswax, and 0-5% frankincense oil. In other embodiments, the foot cream comprises 42.32-52.32% kokum butter, 42.23-52.23% castor oil, 0-10% beeswax, and 0-10% frankincense oil.

Embodiments of the cosmetic compositions in accordance with the present disclosure also include embodiments where said cosmetic composition is a hand cream comprising kokum butter, sesame oil, beeswax, and geranium oil. Collectively, these ingredients provide for an unexpectedly effective hand cream that nourishes, heals, and protects while enveloping the user's hands in a rosy, sweet, and minty scent. In some embodiments, this hand cream comprises 63.5-68.5% sesame oil, 17.5-22.5% kokum butter, 10.84-15.84% beeswax, and 0-5% geranium oil. In other embodiments, this hand cream comprises 61-71% sesame oil, 15-25% kokum butter, 8.34-18.34% beeswax, and 0-10% geranium oil.

Other embodiments of the cosmetic compositions in accordance with the present disclosure exist where said cosmetic composition is an age-defying serum containing sesame oil, sweet almond oil, kokum butter, neroli essential oil and carrot seed essential oil. In these embodiments, the cosmetic composition is a light oil that locks-in moisture, to be used over a film of water on the end user's face. This prolongs the hydration process which in turn plumps out tiny lines on the face of a user. In addition to the benefits provided by kokum butter, sesame oil, and neroli essential old, sweet almond oil is included to help combat free radicals given the high level of antioxidants present in the sweet almond oil. This oil also encourages the production of collages which aids in wrinkle prevention and skin elasticity. Carrot seed oil is included for the carotenoid content, which also provides strong antioxidizing properties. In some embodiments, the age-defying serum comprises 58-63% sesame oil, 25.5-30.5% sweet almond oil, 4.5-9.5% kokum butter, .2-5.2% neroli oil, and 0-5% carrot seed oil. Other embodiments exist where the age-defying serum comprises 55.5-65.5% sesame oil, 23-33% sweet almond oil, 2-12% kokum butter, 0-10% neroli oil, and 0-10% carrot seed oil.

Still other embodiments of the cosmetic compositions in accordance with the present disclosure exist, and such embodiments include a face cleaning jelly containing kokum butter, sesame oil, glycerin, xanthan gum, borax, demineralized water, lavender essential oil, and tea tree essential oil. The cleanser is soothing, anti-fungal, anti-viral and anti-bacterial. In addition to the cleansing benefits, this cleaning jelly erases scars and fine lines, reduces pores and acne, lightens pigmentation and removes a tan, not to mention it softens the skin. Glycerin is included as it is a cleansing agent that is both oil-free and non-comedogenic. Lavender oil is included as it has a calming effect and helps reduce itching, swelling, and redness caused by insect bites, infections, and skin disorders, and provides anti-aging benefits and helps even out skin tone while fighting infections. Tea tree oil is included to improve the antibacterial properties of the cosmetic composition.

One method of creating these embodiments begins by first placing the glycerin and demineralized water together in a vessel where they are heated. Then, the sesame seed oil and kokum butter are placed together in a second vessel and also heated. Both vessels are brought to 40-45 degrees Celsius and then removed from their heat source. The xanthan gum and the borax are mixed will into the water/glycerin solution for roughly one minute while keeping the temperature within 5 degrees from the heated temperature. The two solutions are then quickly mixed together, forming a jelly which is mixed until it has cooled down. Then, the essential oils are added and mixed, completing the embodiment. In some embodiments of this cleaning jelly, the jelly comprises 17-22% sesame oil, 4-9% kokum butter, 17-22% glycerin, 43-48% demineralized water, 0.5-5.5% xanthan gum, 0.5-5.5% borax, 0-5% lavender oil, and 0-5% tea tree oil. In other embodiments of the cleaning jelly, the jelly comprises 14.5-24.5% sesame oil, 1.5-11.5% kokum butter, 14.5-24.5% glycerin, 41.5-51.5% demineralized water, 0-10% xanthan gum, 0-10% borax, 0-10% lavender oil, and 0-10% tea tree oil.

In another embodiment of the cosmetic compositions in accordance with the present disclosure, the cosmetic composition is a lip balm comprising kokum butter, castor oil Jojoba oil, beeswax and aniseed and spearmint essential oils. Jojoba oil is included as it mimics the sebum that is produced naturally in the skin. This not only makes the lips healthier but also helps them to stay hydrated longer. Moreover, this oil creates a barrier so there is less of a chance of the skin becoming chapped. Aniseed oil is included as it alleviate coughs, colds, congestion and bronchitis. Adding this essential oil to these embodiments helps rid the body of these ailments as the person constantly inhales the aroma through the nose and the mouth. Spearmint oil is included as it helps reduce the symptoms of asthma, bronchitis, sinusitis and catarrhal conditions. It has a warm, spicy, herbaceous, minty aroma.

In some embodiments of the lip balm, the lip balm comprises 18.5-23.5% kokum butter, 19.5-24.5% beeswax, 28.5-33.5% castor oil, 19.5-24.5% jojoba oil, 0-5% aniseed oil, and 0-5% spearmint oil. In other embodiments of the lip balm, the lip balm comprises 16-26% kokum butter, 17-27% beeswax, 26-36% castor oil, 17-27% jojoba oil, 0-10% aniseed oil, 0-10% spearmint oil.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” and “third,” are used herein to describe various elements, components, regions, layers and/or sections, these elements, components, regions, layers and/or sections should not be limited by these terms. These terms are only used to distinguish one element, component, region, layer and/or section from another element, component, region, layer and/or section. Thus, a “first element,” “component,” “region,” “layer” and/or “section” discussed below could be termed a second element, component, region, layer and/or section without departing from the teachings herein.

Features illustrated or described as part of one embodiment can be used with another embodiment and such variations come within the scope of the appended claims and their equivalents. Implementations may also include one or a combination of any two or more of the aforementioned features or embodiments.

Spatially relative terms, such as “beneath,” “below,” “lower,” “above,” “upper” and the like, are used herein for case of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the FIGS. is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

Example embodiments are described herein with reference to cross section illustrations that are schematic illustrations of idealized embodiments. As such, variations from the shapes of the illustrations, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

As the invention has been described in connection with what is presently considered to be the most practical and various embodiments, it is to be understood that the invention is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose the invention, including the best mode, and also to enable any person skilled in the art to practice the invention, including making and using any devices or systems and performing any incorporated methods. The patentable scope of the invention is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

The disclosure is illustrated throughout the written description. It should be understood that numerous variations are possible while adhering to the inventive concept. Such variations are contemplated as being a part of the present disclosure.

1. A method of preparing a formulation, the method comprising steps of:
a) forming a first mixture by combining an unrefined plant-based fat with a purified liquid, wherein the purified liquid comprises at least some water content;
b) indirectly heating the first mixture to a temperature in the range of 37 to 40 degrees Celsius;
c) filtering the first mixture through a mesh strainer;
d) solidifying the first mixture by reducing the temperature such that the purified liquid forms a first layer and the plant-based fat forms a second layer, such that the plant-based fat is a refined plant-based fat product;
e) melting the first layer away from the second layer;
f) cutting the refined plant-based fat product into a plurality of pieces;
g) storing the pieces in a fabric package;
h) allowing the refined plant-based fat product within the fabric package to rest at a temperature of 1 to 4 degrees Celsius for a period of at least 24 hours;
i) liquifying the refined plant-based fat product by warming the product to a constant temperature of 100 degrees Celsius;
j) combining at least one oil and at least one substance with the refined plant-based fat product by stirring; and repeating steps b) to e) until the second layer has a substantially homogeneous color.

2. The method of claim 1, wherein the purified liquid is pretreated using at least one of the following methods: chemical treatment, iodine treatment, chlorine treatment, filtration, ultraviolet light, or boiling.

3. The method of claim 1, wherein the purified liquid contains above a 50% water concentration.

4. The method of claim 1, further wherein the unrefined plant-based fat and the purified liquid are combined at a ratio of 4:1 or (80%/20%).

5. The method of claim 1, wherein the plant-based fat is either kokum butter, shea butter, cocoa butter, coconut oil, or mango butter.

6. The method of claim 1, wherein the second layer has a water content equal to or less than 0.20% by total content.

7. The method of claim 1, wherein the plant-based fat and the purified liquid are safely consumable by humans.

8. The method of claim 1, wherein the strainer has a mesh size of 250-44 mm.

9. The method of claim 1, wherein the oil is selected from a group consisting of: coconut oil, sesame oil, essential oil, neroli oil, almond oil, lavender essential oil, tea tree oil, geranium oil, castor oil, frankincense oil, clove bud oil, spearmint oil, jojoba oil, aniseed oil, rose absolute oil, rose oil, clary sage oil, carrot seed oil, and black pepper oil.

10. The method of claim 11, wherein the substance selected from a group consisting of: glycerin, demineralized water, xanthan gum, borax, beeswax, and whole sea salt.

11. A composition, comprising:
a refined plant-based fat product;
a plurality of wood-pressed oils, the wood-pressed oils being selected from the group consisting of: coconut oil, sesame oil, essential oil, neroli oil, almond oil, lavender oil, tea tree oil, geranium oil, castor oil, frankincense oil, clove bud oil, spearmint oil, jojoba oil, aniseed oil, rose absolute oil, rose oil, clary sage oil, and black pepper oil; and
one or more additives, the additives being selected from the group consisting of: glycerin, demineralized water, xanthan gum, borax, beeswax, and whole sea salt.

12. The composition of claim 11,
wherein the plurality of wood-pressed oils are coconut oil, sesame oil, and neroli oil,
wherein the refined plant-based fat product is present in amounts of 32% to 37% w/w,
wherein the coconut oil is present in amounts of 27% to 32% w/w,
wherein the sesame oil is present in amounts of 31% to 36% w/w, and
wherein the neroli oil is present in amounts of 0.5 to 5.5% w/w.

13. The composition of claim 11,
wherein the plurality of wood-pressed oils are coconut oil, sesame oil, and neroli oil,
wherein the refined plant-based fat product is present in amounts of 30% to 40% w/w,
wherein the coconut oil is present in amounts of 20% to 25% w/w,
wherein the sesame oil is present in amounts of 28.5% to 38.5% w/w, and
wherein the neroli oil is present in amounts of up to 10% w/w.

14. The composition of claim 11,
wherein the plurality of wood-pressed oils are sesame oil, lavender oil, and tea tree oil,
wherein the one or more additives are glycerin, demineralized water, xanthan gum, and borax,
wherein the refined plant-based fat product is present in amounts of 4% to 9% w/w,
wherein the sesame oil is present in amounts of 17% to 22% w/w,
wherein the lavender oil is present in amounts of up to 5% w/w,
wherein the tea tree oil is present in amounts of up to 5% w/w,
wherein the glycerin is present in amounts of 17% to 22% w/w,
wherein the xanthan gum is present in amounts of 0.5 to 5.5% w/w,
wherein the borax is present in amounts of 0.5 to 5.5% w/w, and
wherein the demineralized water is present in amounts of 43% to 48% w/w.

15. The composition of claim 11,
wherein the plurality of wood-pressed oils are sesame oil, lavender oil, and tea tree oil,
wherein the one or more additives are glycerin, demineralized water, xanthan gum, and borax,
wherein the refined plant-based fat product is present in amounts of 1.5% to 11.5% w/w,
wherein the sesame oil is present in amounts of 14.5% to 24.5% w/w,
wherein the lavender oil is present in amounts of up to 10% w/w,
wherein the tea tree oil is present in amounts of up to 10% w/w,
wherein the glycerin is present in amounts of 14.5% to 24.5% w/w,
wherein the xanthan gum is present in amounts up to 10% w/w,
wherein the borax is present in amounts of up to 10% w/w, and
wherein the demineralized water is present in amounts of 41.5% to 51.5% w/w.

16. The composition of claim 11,
wherein the plurality of wood-pressed oils are sesame oil, almond oil, neroli oil and carrot seed oil,
wherein the refined plant-based fat product is present in amounts of 4.5% to 9.5% w/w,
wherein the sesame oil is present in amounts of 58% to 63% w/w,
wherein the almond oil is present in amounts of 25.5% to 30.5% w/w,
wherein the neroli oil is present in amounts of 0.2% to 5.2% w/w, and
wherein the carrot seed oil is present in amounts of up to 5% w/w.

17. The composition of claim 11,
wherein the plurality of wood-pressed oils are sesame oil, almond oil, neroli oil and carrot seed oil,
wherein the refined plant-based fat product is present in amounts of 2% to 12% w/w,
wherein the sesame oil is present in amounts of 55.5% to 65.5% w/w,
wherein the almond oil is present in amounts of 23% to 33% w/w,
wherein the neroli oil is present in amounts of up to 10% w/w, and
wherein the carrot seed oil is present in amounts of up to 10% w/w.

18. The composition of claim 11,
wherein the plurality of wood-pressed oils are sesame oil and geranium oil, wherein the one or more additives is beeswax,
wherein the refined plant-based fat product is present in amounts of 17.5% to 22.5% w/w,
wherein the sesame oil is present in amounts of 63.5% to 68.5% w/w,
wherein the geranium oil is present in amounts of up to 5% w/w,
wherein the beeswax is present in amounts of 10.84% to 15.84% w/w.

19. The composition of claim 11,
wherein the plurality of wood-pressed oils are sesame oil and geranium oil,
wherein the one or more additives is beeswax,
wherein the refined plant-based fat product is present in amounts of 15% to 25% w/w,
wherein the sesame oil is present in amounts of 61% to 71% w/w,
wherein the geranium oil is present in amounts of up to 10% w/w,
wherein the beeswax is present in amounts of 8.34% to 18.34% w/w.

20. The composition of claim 11,
wherein the plurality of wood-pressed oils are sesame oil, almond oil, and geranium oil,
wherein the one or more additives are beeswax, demineralized water, borax, and xanthan gum,
wherein the refined plant-based fat product is present in amounts of 17.5% to 22.5% w/w,
wherein the sesame oil is present in amounts of 17.5% to 22.5% w/w,
wherein the almond oil is present in amounts of 17.5% to 22.5% w/w,
wherein the geranium oil is present in amounts of up to 5% w/w,
wherein the beeswax is present in amounts of 17.5% to 22.5% w/w,
wherein the demineralized water is present in amounts of 55.5% to 60.5% w/w,
wherein the borax is present in amounts of up to 5% w/w, and
wherein the xanthan gum is present in amounts of up to 5% w/w.

21. The composition of claim 11,
wherein the plurality of wood-pressed oils are sesame oil, almond oil, and geranium oil,
wherein the one or more additives are beeswax, demineralized water, borax, and xanthan gum,
wherein the refined plant-based fat product is present in amounts of 15% to 25% w/w,
wherein the sesame oil is present in amounts of 15% to 25% w/w,
wherein the almond oil is present in amounts of 15% to 25% w/w,
wherein the geranium oil is present in amounts of up to 10% w/w,
wherein the beeswax is present in amounts of 15% to 25% w/w,
wherein the demineralized water is present in amounts of 53% to 63% w/w,
wherein the borax is present in amounts of up to 10% w/w, and
wherein the xanthan gum is present in amounts of up to 10% w/w.

22. The composition of claim 11,
wherein the plurality of wood-pressed oils are castor oil and frankincense oil,
wherein the one or more additives is beeswax,
wherein the refined plant-based fat product is present in amounts of 44.82% to 49.82% w/w,
wherein the castor oil is present in amounts of 44.73% to 49.73% w/w,
wherein the frankincense oil is present in amounts of up to 5% w/w, and
wherein the beeswax is present in amounts of up to 5% w/w.

23. The composition of claim 11,
wherein the plurality of wood-pressed oils are castor oil and frankincense oil,
wherein the one or more additives is beeswax,
wherein the refined plant-based fat product is present in amounts of 42.32% to 52.32% w/w,
wherein the castor oil is present in amounts of 42.23% to 52.23% w/w,
wherein the frankincense oil is present in amounts of up to 10% w/w, and
wherein the beeswax is present in amounts of up to 10% w/w.
